(19) United States
(12) Patent Application Publication
(10) Pub. No.: US 2008/0051652 A1
(43) Pub. Date: Feb. 28, 2008

(54) ULTRASONIC DIAGNOSTIC APPARATUS AND DIAGNOSTIC METHOD OF THE APPARATUS

(76) Inventors: Kenichi ICHIOKA, Nasushiobara-shi (JP); Muneki Kataguchi, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/753,112

(22) Filed: May 24, 2007

(30) Foreign Application Priority Data
Jun. 1, 2006 (JP) 2006-153574

(51) Int. Cl. A61B 8/00

(52) U.S. Cl. 600/437

(57) ABSTRACT

An ultrasonic diagnostic apparatus of the present invention capable of operation with a three-dimensional ultrasonic image performs a scan with a 3D probe for a predetermined time to acquire 3D volume data, and displays a plurality of sections of a heart on a monitor display, in conducting a functional diagnosis of the motion of the heart walls of a specimen before and after the application of a load. Then, the ultrasonic diagnostic apparatus displays a positional relation between the probe and the heart so that display angles of the sections of the heart displayed on the monitor display may be constant.

### CROSS-REFERENCE TO RELATED APPLICATIONS

This application is based upon and claims the benefit of priority from prior Japanese Patent Application No. 2006-153574, filed Jun. 1, 2006, the entire contents of which are incorporated herein by reference.

### BACKGROUND OF THE INVENTION

1. Field of the Invention

The present invention relates to an ultrasonic diagnostic apparatus and a diagnostic method of the apparatus, and more particularly, it relates to an ultrasonic diagnostic apparatus which scans a section of a specimen with an ultrasonic beam and obtains a three-dimensional image and which thus improves the efficiency of three-dimensional image collection/inspection.

2. Description of the Related Art

In an ultrasonic diagnosis, there is a diagnostic method called stress echocardiography which is a functional diagnosis of heart wall motion before and after the application of a load. This method comprises, for example, causing a specimen to jog, taking images of the myocardium before and after jogging, and comparing these images to determine an abnormal region. Moreover, there is a function to display wall motion scores of the stress echocardiography in accordance with a format.

During a diagnosis with a two-dimensional (2D) cross-sectional image, an operator adapts the cross-sectional image itself to the format in order to display various regions, and an apparatus displays a functional evaluation regarding the cross-sectional image in a divided manner in accordance with the format (e.g., refer to Jpn. Pat. Appln. KOKAI Publication No. 2004-313551).

However, in a recently developed three-dimensional (3D) diagnosis, section perpendicular to an ultrasonic beam are obtained from volume data acquired by a scan for a given time from cardiac apex approach in order to form a so-called C mode view. In a method of scoring the stress echocardiography, much attention should be paid in adjusting the vertical and horizontal positions of the image to the stress echocardiographic scheme due to the influence of the scanning direction of a probe, the movement of the image, etc.

On the other hand, in order to make use of the fact that an arbitrary 2D cross-sectional image can be analyzed after one scan owing to the volume data which characterizes the 3D, it is necessary to be able to display a C mode view adapted to the format of the scoring of the stress echocardiography. When an image acquired by the operator is not the C mode view adapted to the format, the rotation, etc. of the image is required to adapt the image to the format. There is therefore a problem that a short scan time does not result in a reduction of a diagnostic time due to the following format adaptation.

### BRIEF SUMMARY OF THE INVENTION

It is therefore an object of the present invention to provide an ultrasonic diagnostic apparatus and a diagnostic method of this apparatus, wherein scores of stress echocardiography are displayed in accordance with a predetermined report format from 3D volume data, and there is thus no need for the rotation, etc. of an acquired image, leading to an improvement of throughput and reduction in inspection time, such that burdens on a patient and an operator can be reduced.

The present invention therefore provides

an ultrasonic diagnostic apparatus configured to perform an ultrasonic scan of a three-dimensional region, the apparatus comprising:

three-dimensional data acquisition unit configured to acquiring three-dimensional volume data;

display unit configured to displaying an ultrasonic cross-sectional image of an arbitrary position included in the three-dimensional region; and

guide information generating unit configured to generating guide information to guide a positional relation between a probe and a specimen so that the displayed ultrasonic cross-sectional image shows a section of a predetermined position in the specimen,

wherein the display unit displays the guide information together with the ultrasonic cross-sectional image of the section of the arbitrary position.

The present invention also provides

an ultrasonic diagnostic method which performs an ultrasonic scan of a three-dimensional region, the method comprising the steps of:

acquiring three-dimensional volume data and displaying a plurality of sections of a specimen; and

reporting a positional relation between a probe and the specimen so that the displayed sections of the specimen show sections of the specimen in a predetermined form.

According to the present invention, it is possible to provide an ultrasonic diagnostic apparatus and a diagnostic method of this apparatus, wherein scores of stress echocardiography are displayed in accordance with a predetermined report format from 3D volume data, and there is thus no need for the rotation, etc. of an acquired image, leading to an improvement of throughput and reduction in inspection time, such that burdens on a patient and an operator can be reduced.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing a schematic configuration of an ultrasonic diagnostic apparatus in a first embodiment of the present invention;

FIG. 2A is a diagram showing an example of the position of a probe during echocardiography using a general two-dimensional scanning ultrasonic diagnostic apparatus, and FIG. 2B is a diagram showing a relation with a basic cross-sectional image conforming to the position of the probe in FIG. 2A;

FIGS. 3A to 3C show examples of the basic cross-sectional image in the echocardiography on various levels shown in FIG. 2B, wherein FIG. 3A is a diagram showing a short axis view of left ventricle at the left sternal border on a cardiac apex level, FIG. 3B is a diagram showing a short axis view of left ventricle at the left sternal border on a papillary muscle level, and FIG. 3C is a diagram showing a short axis view of left ventricle at the left sternal border on a mitral orifice level;

FIG. 4 is a flowchart for explaining a procedure of a diagnosis by the ultrasonic diagnostic apparatus in the first embodiment of the present invention;

FIG. 5 is a diagram showing an example of how to search for a four-chamber cross-sectional image through cardiac apex approach by use of the ultrasonic diagnostic apparatus having the configuration in FIG. 1;

FIGS. 6A and 6B show the relation of three sections through cardiac apex approach, wherein FIG. 6A is a diagram showing an arrangement relation between a heart and a 3D probe, and FIG. 6B is a diagram showing three sectional lines in a C mode view;

FIGS. 7A to 7C show examples of the basic cross-sectional image in the echocardiography, wherein FIG. 7A is a four-chamber sectional image through cardiac apex approach, FIG. 7B is a two-chamber sectional image through cardiac apex approach, and FIG. 7C is a diagram showing a long axis view of left ventricle through cardiac apex approach;

FIGS. 8A to 8G show examples of scoring formats of stress echocardiography, wherein FIG. 8A is a short axis view of parasternal left ventricle and is a diagram showing the base of a heart, FIG. 8B is a short axis view of parasternal left ventricle and is a diagram showing a central part thereof, FIG. 8C is a short axis view of parasternal left ventricle and is a diagram showing cardiac apex, FIG. 8D is a diagram showing a long axis view of parasternal left ventricle, FIG. 8E is a diagram showing a two-chamber view of cardiac apex, FIG. 8F is a diagram showing a four-chamber view of cardiac apex, and FIG. 8G is a diagram showing a long axis view of cardiac apex;

FIG. 9 is a diagram showing an example of monitor display layout for the recognition of a positional relation between a 3D probe 22 and a heart 50;

FIG. 10A is a diagram showing information for urging the display of a two-chamber cross-sectional image, and FIG. 10B is a diagram showing information for urging the display of a long axis cross-sectional image; and

FIG. 11 is a diagram showing an example of monitor display layout for the recognition of a positional relation between the 3D probe 22 and the heart 50 in a second embodiment of the present invention.

### DETAILED DESCRIPTION OF THE INVENTION

Hereinafter, embodiments of the present invention will be described with reference to the drawings.

### First Embodiment

Initially, a first embodiment of the present invention will be described.

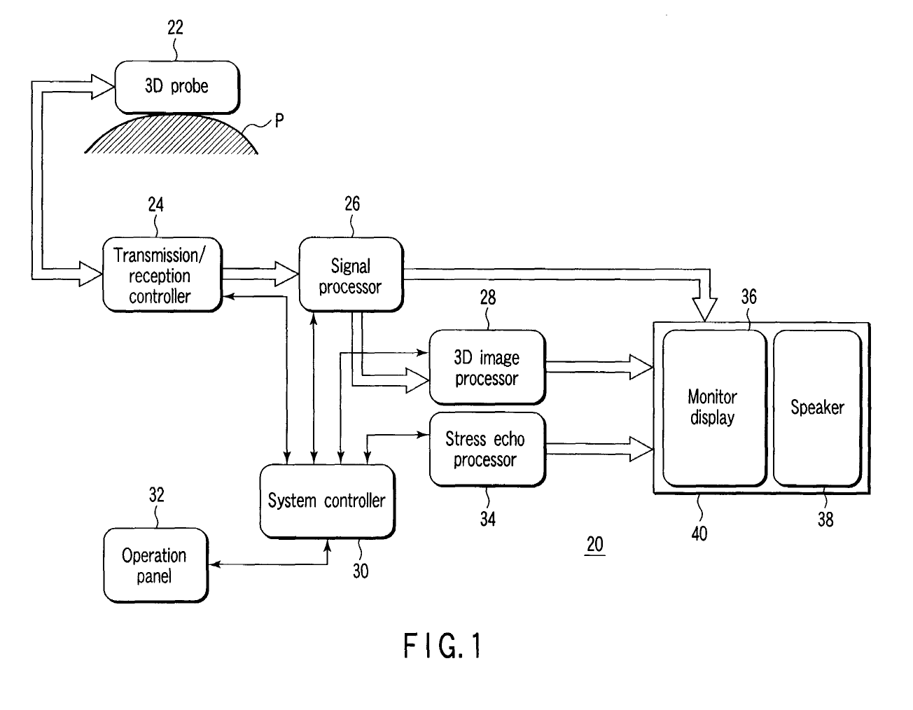

FIG. 1 is a block diagram showing a schematic configuration of an ultrasonic diagnostic apparatus in a first embodiment of the present invention.

In FIG. 1, an ultrasonic diagnostic apparatus 20 comprises a three-dimensional ultrasonic probe (3D probe) 22, a transmission/reception controller 24 including a transmission/reception unit, a signal processor 26, a 3D image processor 28, a system controller 30, an operation panel 32, a stress echo processor 34, and an output unit 40 having a monitor display 36 and a speaker 38.

The 3D probe 22 sends/receives ultrasonic waves to/from a specimen P to obtain an ultrasonic cross-sectional image, and the transmission/reception controller 24 transmits/receives an electric signal to/from the 3D probe 22. The signal processor 26 processes a transmission/reception signal obtained from the transmission/reception controller 24, and generates and stores three-dimensional volume data, etc. The 3D image processor 28 generates, from data generated in the signal processor 26, a 3D image to be displayed on the monitor display 36, and also stores the 3D view. Then, the monitor display 36 in the output unit 40 displays the image data, etc. generated in the signal processor 26 and the 3D image processor 28.

Furthermore, the system controller 30 controls the entire ultrasonic diagnostic apparatus. The system controller 30 also extracts a section of a desired position of the specimen from the stored three-dimensional volume data on the basis of the positional relation between the three-dimensional volume data and the ultrasonic cross-sectional image, assuming that the ultrasonic cross-sectional image shows a section of a predetermined position. The operation panel 32 is provided for an operator to operate the 3D probe and to input information. Further, the stress echo processor 34 generates and stores an image which is obtained by stress echocardiography and which is to be displayed on the monitor display 36. Moreover, the speaker 38 in the output unit 40 generates voice guides, sound effects, etc. stored in an unshown memory or the like in the system controller 30, when the operator operates the operation panel 32.

Here, an example of a basic cross-sectional image in echocardiography by general two-dimensional (2D) scanning will be described.

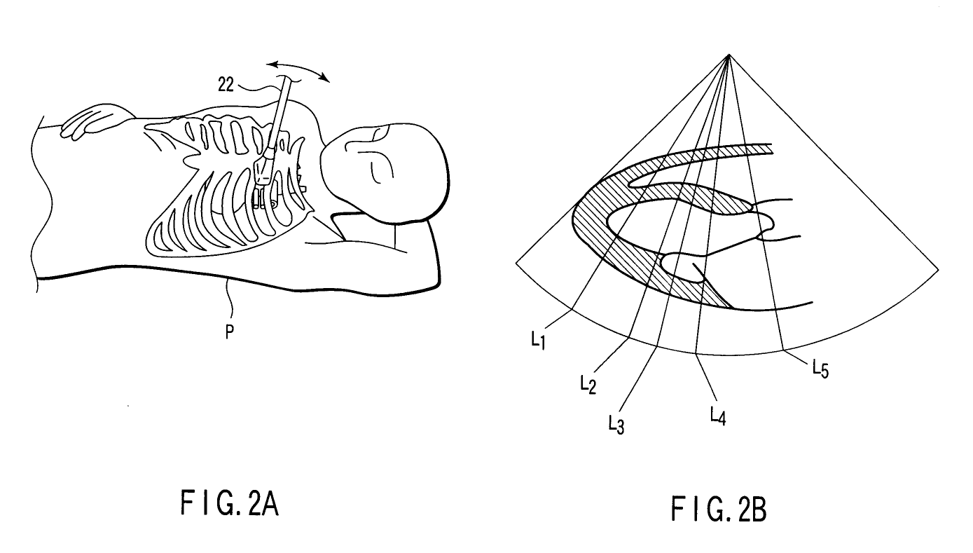

FIGS. 2A and 2B are diagrams showing the relation between the position of the probe during echocardiography using the ultrasonic diagnostic apparatus and the basic cross-sectional image by the 2D scanning. Now, as shown in FIG. 2A, the operator places the 3D probe 22 over the heart of the specimen P, and changes the inclination of the probe in a direction indicated by an arrow. Then, a short axis view on each level is extracted by the inclination, as shown in FIG. 2B. In this case, in a long axis cross-sectional image of left ventricle, L₁ shows a cross-sectional image on a cardiac apex level, L₂ shows a cross-sectional image on a papillary muscle level, L₃ shows a cross-sectional image on a tendinous chord level, L₄ shows a cross-sectional image on a mitral orifice level, and L₅ shows a cross-sectional image on an aortic valve level.

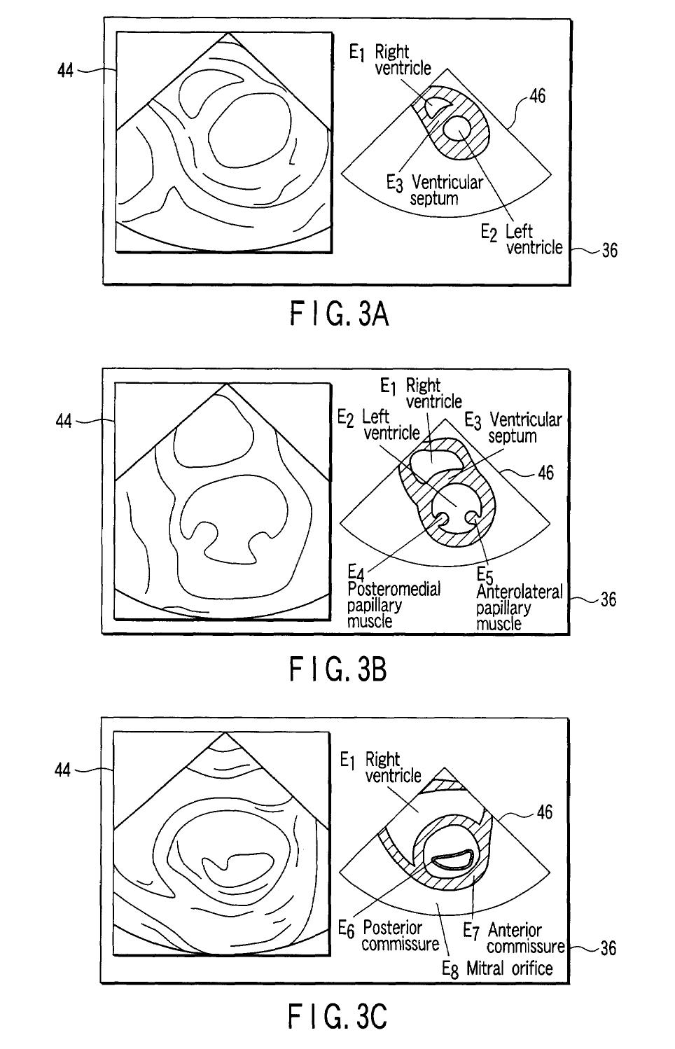

FIGS. 3A to 3C show examples of the basic cross-sectional image in the echocardiography on various levels shown in FIG. 2B, wherein FIG. 3A is a diagram showing a short axis view of left ventricle at the left sternal border on the cardiac apex level L₁, FIG. 3B is a diagram showing a short axis view of left ventricle at the left sternal border on the papillary muscle level L₂, and FIG. 3C is a diagram showing a short axis view of left ventricle at the left sternal border on the mitral orifice level L₄. In addition, in each of FIGS. 3A to 3C, the left side of the screen of the monitor display 36 shows a cross-sectional image 44 by actual stress echocardiography, and the right side of the screen shows an explanatory view 46 indicating regions of the cross-sectional image 44. Moreover, indications E₁ to E₈ in the explanatory view 46 indicate the regions in this explanatory view.

When such a cross-sectional image is used, the rotation, etc. of the image have heretofore been needed to adapt the image to the format of the scoring of the stress echocardiography if this image is not adapted thereto.

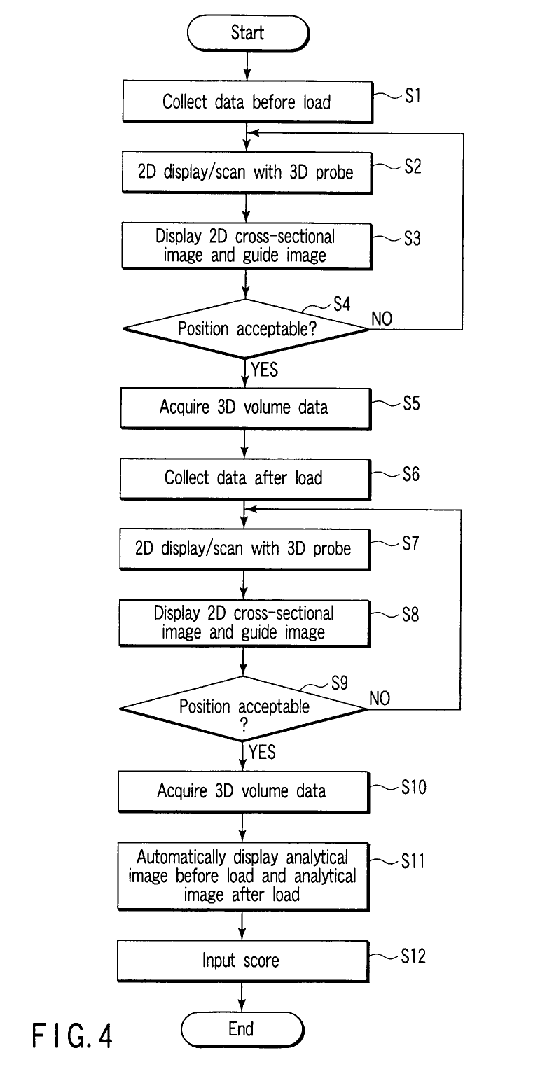

Next, a procedure of a diagnosis by the ultrasonic diagnostic apparatus in the present embodiment will be described in accordance with a flowchart in FIG. 4.

First, data before the application of a load is collected in step S1, and a 2D display/scan with the 3D probe 22 is performed in step S2.

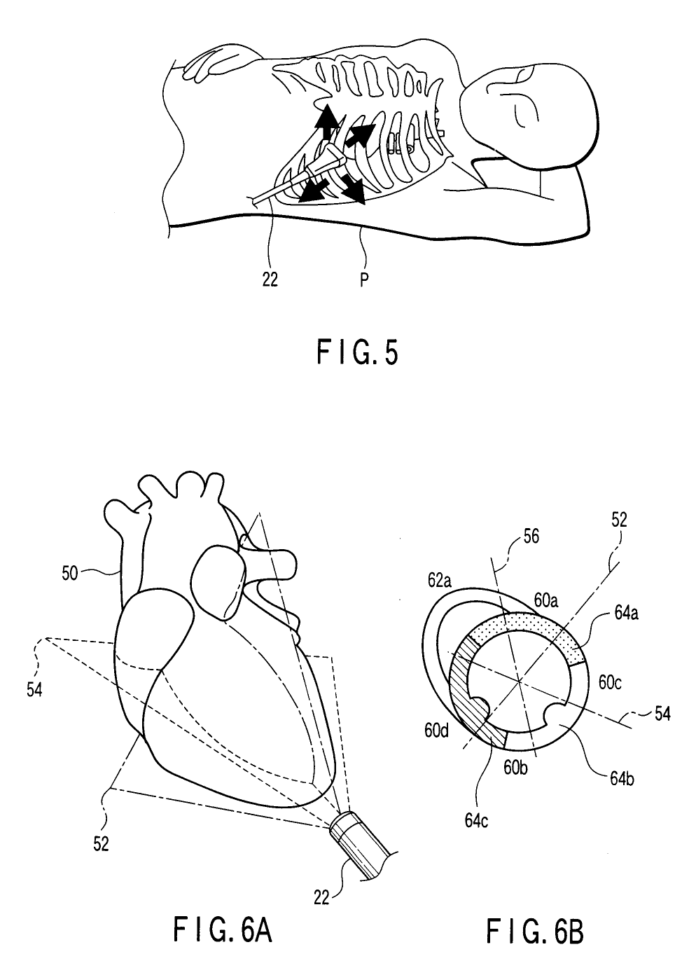

FIG. 5 is a diagram showing an example of how to search for a four-chamber cross-sectional image through cardiac apex approach by use of the ultrasonic diagnostic apparatus according to the first embodiment of the present invention.

In the ultrasonic diagnostic apparatus having the configuration described above, the operator searches the heart of the specimen P for an apex beat from the direction of cardiac apex as indicated by arrows, and places the 3D probe 22 thereon. FIGS. 6A and 6B are diagrams showing the relation of three sections through cardiac apex approach obtained in this manner. While no sectional line of a long axis view is shown in FIG. 6A, three sectional lines in a C mode view are shown in FIG. 6B. That is, when the 3D probe 22 is disposed in the direction of the cardiac apex of a heart 50, a two-chamber view 52, a four-chamber view 54 and a long axis view 56 of the heart 50 can be obtained in accordance with the angle of the 3D probe 22. In FIG. 6B, 60a denotes an anterior wall, 60b denotes a posterior wall, 60c denotes a lateral wall, 60d denotes an inferior wall, 62a denotes an anterior wall septum, 62b denotes an inferior wall septum, 64a denotes left anterior descending branch perfusion, 64b denotes left circumflex branch perfusion, and 64c denotes right coronary artery perfusion.

In step S3, a 2D cross-sectional image and a guide image obtained in step S2 are displayed on the monitor display 36. In step S4, the position where the 3D probe 22 is placed is checked. As a result, the transition is made to step S2 if the position of the 3D probe 22 is not acceptable, and operations in steps S2 to S4 are repeated until step S4 accepts the position. Then, when the position is accepted in step S4, the transition is made to step S5, and 3D volume data by the 3D probe 22 is acquired.

Next, data after the application of the load is collected in step S6, and a 2D display/scan with the 3D probe 22 is performed in step S7. Then, in step S8, a 2D cross-sectional image and a guide image after the load obtained in step S7 are displayed on the monitor display 36. In step S9, the position where the 3D probe 22 is placed is checked. As a result, the transition is made to step S7 if the position of the 3D probe 22 is not acceptable, and operations in steps S2 to S4 are repeated. Then, when the position is accepted in step S9, the transition is made to step S10, and 3D volume data after the load is acquired.

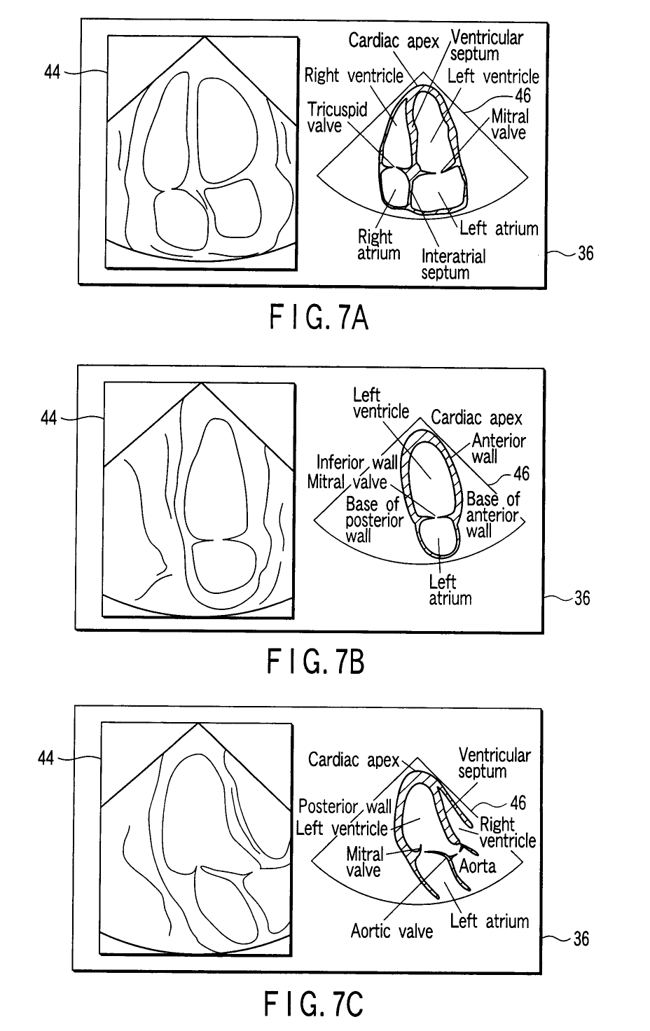

FIGS. 7A to 7C show a four-chamber sectional image through cardiac apex approach, a two-chamber sectional image through cardiac apex approach and a long axis view of left ventricle through cardiac apex approach, respectively. In addition, in each of FIGS. 7A to 7C, the left side of the screen of the monitor display 36 shows the cross-sectional image 44 by actual stress echocardiography, and the right side of the screen shows the explanatory view 46 indicating the regions of the cross-sectional image 44. Thus, the positional relation of the heart is decided on the basis of the angle of inclination (position) of the 3D probe 22 as shown in FIG. 6A.

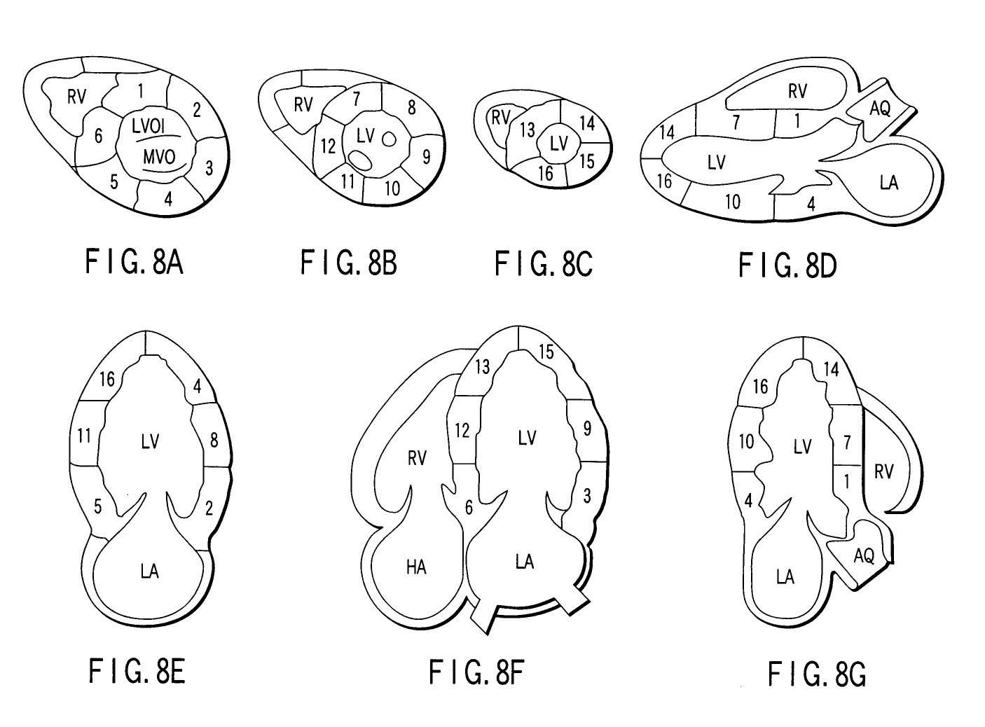

FIGS. 8A to 8G are diagrams showing examples of scoring formats of stress echocardiography. FIGS. 8A to 8C show short axis views of parasternal left ventricle, wherein FIG. 8A is a diagram showing the base of the heart, FIG. 8B is a diagram showing a central part thereof, and FIG. 8C is a diagram showing cardiac apex. Further, FIG. 8D is a diagram showing a long axis view of parasternal left ventricle, FIG. 8E is a diagram showing a two-chamber view of cardiac apex, FIG. 8F is a diagram showing a four-chamber view of cardiac apex, and FIG. 8G is a diagram showing a long axis view of cardiac apex.

As shown in the drawings, the intracardiac left ventricle is classified into segments, and numbers are assigned to these segments, in order to quantify the degree of abnormality segment by segment. In this case, the left ventricle is divided into sixteen segments indicated by 1 to 16 on the basis of four cross sections including a short axis section, a long axis section, a four-chamber section and a two-chamber section. Thus, the degrees of abnormality in heart wall motion in the scoring are displayed so that the segments are color-coded in accordance with the degrees of abnormality.

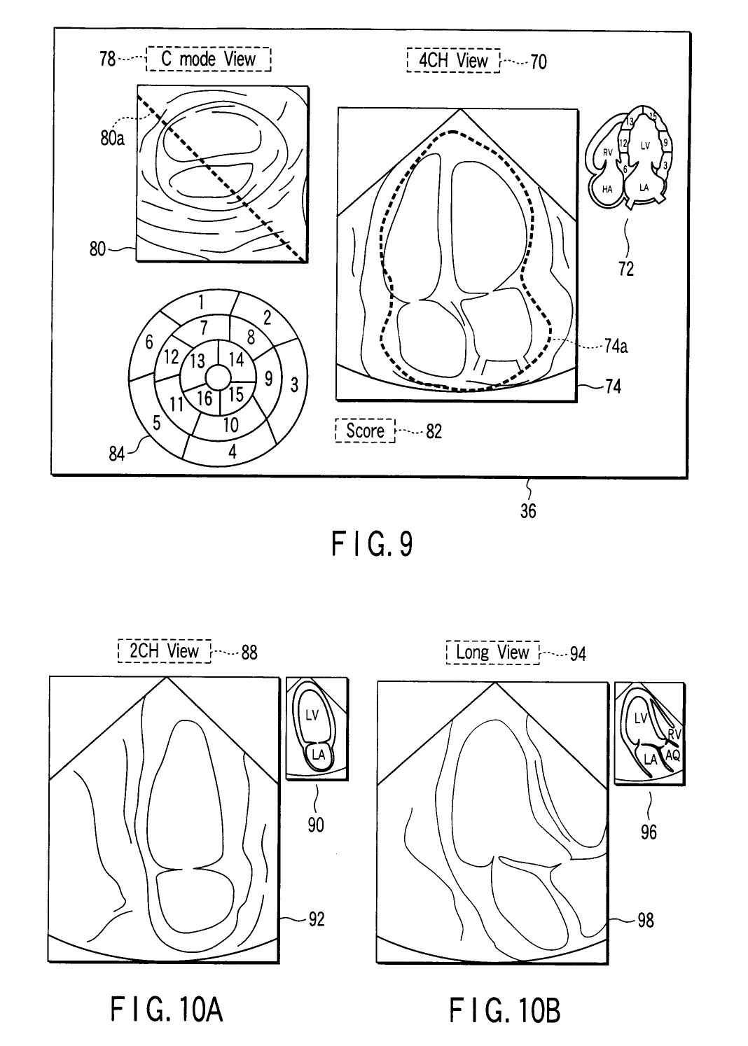

As described above, the operator scans the whole heart for a given time through cardiac apex approach as shown in FIG. 6A to acquire the volume data. At this point, the apparatus recognizes the positional correlation between the heart and the probe to adapt the C mode view to the scoring formats as shown in FIGS. 8A to 8G. Therefore, information for urging the display of a four-chamber cross section as shown in FIG. 9 is provided on the monitor display 36.

That is, in step S11, an analytical image before the application of the load and an analytical image after the application of the load are displayed on the monitor display 36. Then, in step S12, the operator observes the analytical images before and after the application of the load displayed on the monitor display 36 to input into a scoring pattern in the color-coded manner described above on the basis of differences between these images.

FIG. 9 is a diagram showing an example of monitor display layout for the recognition of a positional relation between the 3D probe 22 and the heart 50. That is, the monitor display 36 shows the C mode view of the heart through cardiac apex approach as shown in FIG. 6A, the position of a section though this C mode view, a sectional image (a four-chamber view in FIG. 9) obtained from this position of the section, and the scoring pattern.

In the case of FIG. 9, the information for urging the display of the four-chamber cross-sectional image is provided. The information provided on the screen of the monitor display 36 includes character information (indicated as “4CH view”) 70 for urging the display of the four-chamber cross-sectional image, pictographic (body marker) information 72 for urging the display of the four-chamber cross-sectional image, and an outline 74a as ROI information 74 for urging the display of the four-chamber cross-sectional image. Since the outline 74a is the analytical image before the load, the analytical image after the load is adapted to this outline 74a to adjust the position of the probe. Moreover, other information for urging the display of the four-chamber cross-sectional image includes, for example, the voice guides and the sound effects generated from the speaker 38.

The present embodiment permits one of such information for urging the display of the four-chamber cross-sectional image to be provided, or more than one of them to be simultaneously provided.

Furthermore, there are shown, on the screen of the monitor display 36, character information (indicated as “C mode view”) 78 indicating that a display image 80 is in a C mode display, the C mode display image 80 including a section position marker 80a of the cross-sectional image displayed by the ROI information 74, character information (indicated as “score”) 82 meaning the indication of a scoring display, and a scoring pattern 84. This scoring pattern 84 corresponds to the numbers of the segments shown in FIGS. 8A to 8G. Although colors are not displayed in FIG. 9, the degrees of abnormality in the respective segments can be actually recognized by the color-coded display as described above.

Moreover, although not shown in FIG. 9, a section position marker may also be displayed on the scoring pattern 84.

For example, suppose that the operator is attempting to display a four-chamber cross-sectional image on the monitor screen. At this point, as shown in FIG. 9, characters and an image for urging the display of the four-chamber cross-sectional image are displayed by the character information 70 and the body marker information 72, and the image is compared with the actual ROI information 74 (outline 74a). The result of this comparison proves whether a desired four-chamber cross-sectional image is correctly obtained. Further, the scoring pattern 84 makes it possible to easily recognize by color which region within the heart is displayed and the condition in that region. For example, the scoring pattern 84 indicates a normal state in green and an abnormal state in red. It is to be noted that the colors displayed by the scoring pattern 84 and the number of colors are not limited thereto.

As described above, the operator places the 3D probe 22 in the vicinity of the heart 50 of the specimen P, and changes the inclination of the 3D probe 22, such that a desired cross-sectional image (a four-chamber cross-sectional image in the example of FIG. 9) and its associated information are displayed on the monitor display 36. In this case, the four-chamber cross-sectional image can be identified not only by the ROI information 74 but also by the character information 70, the pictographic information 72, the section position marker 80a in the C mode view 80, etc. Then, when the image is adapted to the various kinds of information displayed on the monitor display 36, especially to the outline 74a of the ROI information 74, the position of the probe is adjusted, and the operator can correctly obtain the desired cross-sectional image.

Thus, because the positional relation of the heart is decided on the basis of the angle of inclination of the 3D probe 22, the operator does not have to rotate the image acquired from the specimen P in accordance with the information provided on the monitor display 36, so that throughput can be improved.

Furthermore, while the display of the four-chamber cross-sectional image is explained as an example in the embodiment described above, it should be understood that the present invention is not limited to this.

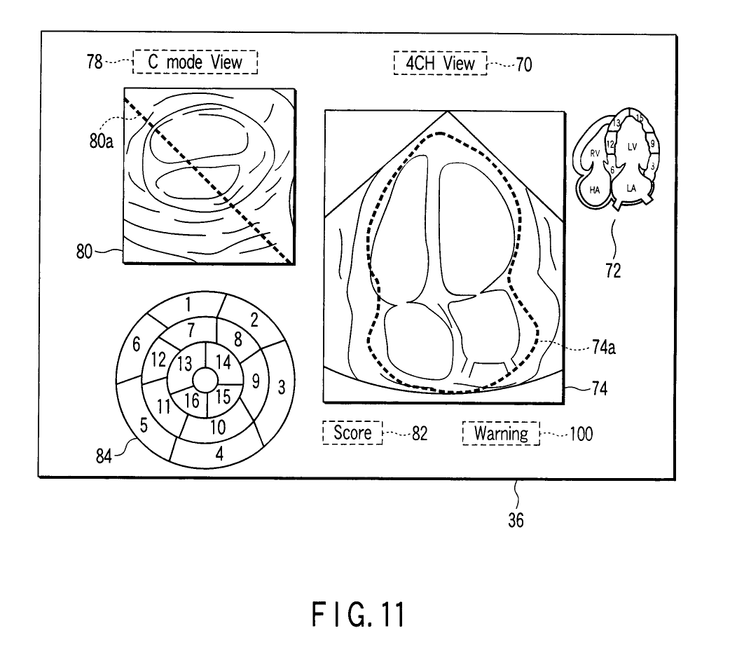

For example, as shown in FIG. 10A, information for urging the display of the two-chamber cross-sectional image may be provided. That is, suppose that a cross-sectional image obtained from the position of a section through the C mode view of the heart through cardiac apex approach as shown in FIG. 6A is a two-chamber cross-sectional image. In this case, provided information includes character information (indicated as “2CH view”) 88 for urging the display of the two-chamber cross-sectional image, pictographic (body marker) information 90 for urging the display of the two-chamber cross-sectional image, and ROI information 92 for urging the display of the two-chamber cross-sectional image. Such information is provided on the screen of the monitor display 36 in place of the character information 70, the pictographic information 72 and the ROI information 74. Further, the section position marker 80a within the C mode view shown in FIG. 9 and the section position marker that can be displayed in the scoring pattern 84 are displayed at the position of the section through the two-chamber cross-sectional image.

Furthermore, as shown in FIG. 10B, information for urging the display of a long axis cross-sectional image may be provided. In this case, if a cross-sectional image obtained from the position of a section through the C mode view of the heart through cardiac apex approach as shown in FIG. 6A is a long axis cross-sectional image, provided information includes character information (indicated as “long view”) 94 for urging the display of the long axis cross-sectional image, pictographic (body marker) information 96 for urging the display of the long axis cross-sectional image, and ROI information 98 for urging the display of the long axis cross-sectional image. These are provided on the screen of the monitor display 36 in place of the character information 70, the pictographic information 72 and the ROI information 74. Further, the section position marker 80a within the C mode view shown in FIG. 9 and the section position marker that can be displayed in the scoring pattern 84 are displayed at the position of the section through the long cross-sectional image.

Thus, in any of the cross-sectional images, it is possible to accurately know the positional relation between the 3D probe and the heart by the character information, the pictographic (body marker) information, the ROI information, sound information, etc. for urging the display of the cross-sectional image.

It is to be noted that the information for urging the display of the cross-sectional image described above and the kind of information can also be selected by the operation on the operation panel 32.

### Second Embodiment

While the positional relation between the 3D probe and the heart is indicated in the first embodiment described above, a warning is issued when a cross-sectional image is not correctly scanned, in a second embodiment.

In addition, in the present second embodiment, the configuration and basic operation of an ultrasonic diagnostic apparatus are the same as the configuration and operation of the ultrasonic diagnostic apparatus in the first embodiment shown in FIGS. 1 to 10A and 10B. Therefore, the same reference numerals are assigned to the same parts, and different parts alone will be described without diagrammatically showing and describing the same parts.

FIG. 11 is a diagram showing an example of monitor display layout for the recognition of a positional relation between a 3D probe 22 and a heart 50 in the second embodiment of the present invention. Here, a warning sign (e.g., “warning”) 100 is provided on a screen as to whether a four-chamber cross-sectional image obtained as ROI information 74 is a correct cross-sectional image. Whether the four-chamber cross-sectional image is a correct cross-sectional image is judged in accordance with, for example, pattern recognition by, for example, a pattern recognition unit within a system controller 30. When the cross-sectional image is judged as one different from the correct cross-sectional image as a result, the above-mentioned warning sign 100 is provided on the screen of a monitor display 36.

Pictographic information 72 and the ROI information 74 (an outline 74a) are provided as warning information including character information 70 for urging a warning such as the warning sign 100. These are displayed in a flashing manner and thus recognized as a warning. Alternatively, voice guides and sound effects may be generated to send a warning through a speaker 38.

In the present embodiment, one of such warning information may be provided, or more than one of such warning information may be simultaneously provided. Moreover, the information and the kind of information can be selected by, for example, the operation of an operation panel 32.

While the embodiments of the present invention have been described above, the present invention is not limited to the embodiments described above, and various modification can be made without departing from the spirit of the present invention.

Furthermore, the embodiments described above include inventions at various stages, and suitable combinations of a plurality of disclosed constitutional requirements permit various inventions to be extracted. For example, when the problems described in the section BACKGROUND OF THE INVENTION can be solved and the advantages described in the section BRIEF SUMMARY OF THE INVENTION can be obtained even if some of all the constitutional requirements shown in the embodiments are eliminated, a configuration in which those constitutional requirements are eliminated can also be extracted as an invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic diagnostic apparatus configured to perform an ultrasonic scan of a three-dimensional region, the apparatus comprising:
three-dimensional data acquisition unit configured to acquiring three-dimensional volume data;
display unit configured to displaying an ultrasonic cross-sectional image of an arbitrary position included in the three-dimensional region; and
guide information generating unit configured to generating guide information to guide a positional relation between a probe and a specimen so that the displayed ultrasonic cross-sectional image shows a section of a predetermined position in the specimen,
wherein the display unit displays the guide information together with the ultrasonic cross-sectional image of the section of the arbitrary position.

2. The ultrasonic diagnostic apparatus according to claim 1, wherein
the guide information generating unit generates a score of a functional diagnosis of heart wall motion corresponding to the section of the predetermined position.

3. The ultrasonic diagnostic apparatus according to claim 2, wherein
the display unit simultaneously displays the ultrasonic cross-sectional images of a plurality of sections, and
the guide information generating unit generates a score corresponding to each of the plurality of ultrasonic cross-sectional images.

4. The ultrasonic diagnostic apparatus according to claim 1, wherein
the guide information generating unit generates the guide information, regarding a short axis section or long axis section of the heart as the section of the predetermined position.

5. The ultrasonic diagnostic apparatus according to claim 1, wherein
the guide information generating unit generates a schematic image which shows a schematic form of the section of the predetermined position and which has a size corresponding to the ultrasonic cross-sectional image, and
the display unit displays the schematic image so that this schematic image is superposed on the ultrasonic cross-sectional image.

6. The ultrasonic diagnostic apparatus according to claim 5, further comprising:
detection unit configured to detecting the degree of coincidence between the schematic image and the ultrasonic cross-sectional image; and
report unit configured to providing an operator with a report corresponding to the result of the detection by the detection means.

7. The ultrasonic diagnostic apparatus according to claim 1, further comprising:
storage unit configured to storing the three-dimensional volume data on the basis of an operation by the operator; and
section extracting unit configured to extracting a section of a desired position of the specimen from the stored three-dimensional volume data on the basis of the positional relation between the three-dimensional volume data and the ultrasonic cross-sectional image, assuming that the ultrasonic cross-sectional image shows the section of the predetermined position.

8. The ultrasonic diagnostic apparatus according to claim 6, wherein
the report unit reports at least one of character information, pictographic information, ROI information and sound information.

9. The ultrasonic diagnostic apparatus according to claim 6, further comprising:
selection unit configured to selecting the kind of information reported by the report unit.

10. The ultrasonic diagnostic apparatus according to claim 1, wherein
the three-dimensional volume data is acquired by stress echocardiography.

11. An ultrasonic diagnostic method which performs an ultrasonic scan of a three-dimensional region, the method comprising the steps of:
acquiring three-dimensional volume data and displaying a plurality of sections of a specimen; and
reporting a positional relation between a probe and the specimen so that the displayed sections of the specimen show sections of the specimen in a predetermined form.
